(19) United States
(12) Patent Application Publication
(10) Pub. No.: US 2012/0252754 A1
(43) Pub. Date: Oct. 4, 2012

(54) Candy Cakes

(75) Inventor: Holly Frances Miller, Centralia, MO (US)

(73) Assignee: Holly Frances Miller, Centralia, MO (US)

(21) Appl. No.: 13/437,922

(22) Filed: Apr. 2, 2012

Related U.S. Application Data
(60) Provisional application No. 61/512,196, filed on Jul. 27, 2011.
(60) Provisional application No. 61/516,196, filed on Mar. 31, 2011.

(51) Int. Cl. A61K 31/7016; A61P 3/12

(52) U.S. Cl. 514/53

(57) ABSTRACT

Hyperkalemic Periodic Paralysis is a disease in mainly stock type horses that can be treated by giving the horse light corn syrup. Currently, the easiest way to do this is with a syringe, which is actually very difficult. The invention is a treat that will deliver a specific amount of light corn syrup per treat. This makes the entire process of delivering the corn syrup easier, faster, cleaner, and actually enjoyable for the horse. The treat can be used as an abortive measure, and also as a preventative measure if the owner incorporates the treat into the horses' daily routine. In addition to being used as the treatment for the disease, it can also be used as a regular treat for HYPP positive horses.

### CROSS REFERENCE TO RELATED APPLICATION

This patent application corresponds with provisional patent application U.S. 61/516,196, filed Mar. 31, 2011, under applicant name Holly Frances Miller, with title of invention ‘Candy Cakes’.

### STATEMENT REGARDING FEDERALLY SPONSERED RESEARCH OR DEVELOPMENT

Not applicable cl REFERENCE TO SEQUENCE LISTING, A TABLE, OR A COMPUTER PROGRAM LISTING COMPACT DISK APPENDIX

Not applicable

### BACKGROUND OF THE INVENTION

Hyperkalemic Periodic Paralysis is a disease that can affect any horse descending from the American Quarter Horse stallion Impressive. The disease is a dominant disease, meaning only one copy of the gene is necessary for the animal to be affected. The gene sequences are referred to as: N/N-doesn't have a copy and isn't affected, N/H-has one copy and therefore can be affected, and H/H-double positive and affected. The symptoms vary in severity, though H/H horses are often more severely affected. Common symptoms are muscle twitching, typically towards the hind end, generalized weakness, and difficulty breathing, resulting in a roaring sound. HYPP can also be confused with other issues such as colic or tying up, because of the seemingly similar symptoms (UC Davis, 2009.)

The specifics of the disease are related to potassium and sodium channels within the cells. During exercise, the cells have more sodium inside the cell and more potassium outside the cell. With an attack, the sodium channels leak sodium into the muscles, which causes potassium to move into the bloodstream (HYPP, 2010.)

All studies and literature are in agreement as to what is the best prevention and treatment for HYPP positive horses. The general consensus is these horses should be kept on the lowest potassium diet possible. The maximum daily potassium intake should be no higher than 1.5%. This can be done through feeding low potassium feeds, such as oats, beet pulp, or timothy hay, several times a day. High potassium feeds such as alfalfa hay, soybean meal, and sweet feeds with molasses should be avoided. Exercise is another very important step to preventing attacks.

Administering diuretics every 12 hours, such as hydrochlorothiazide or acetazolamide, can be very helpful as well (UC Davis, 2009.)

During minor attacks, the horse should be given grain and light corn syrup, both of which increase insulin. Insulin is very important, as it stimulates the cells to bring potassium back into the cells, and out of the bloodstream. Exercise increases adrenaline, which also helps with putting potassium back into the cells. Administration of hydrochlorothiazide or acetazolamide helps the horse get rid of potassium through waste. Severe attacks should involve the vet immediately (Sellnow, 2001.) The main problem with current treatment using light corn syrup in a syringe is that it is difficult, messy, and time consuming.

References
Dehn, C. A., & Prien, S. L. (2007, Oct. 11). Substance and process for preventing a genetic defect from being passed onto offspring. Retrieved from http://www.freepatentsonline.com/20070238680.pdf
Hyperkalemic Periodic Paralysis. (2009). UC Davis Veterinary Medicine online. Retrieved from http://www.vgl.ucdavis.edu/services/hypp.php
HYPP. (January 2010.) Westphalia Ranch. Retrieved from http://www.westphalia-ranch.com/hypp.htm
Sellnow, L. (2001, 10 Oct.). HYPP: Hyperkalemic Periodic Paralysis. The Horse, article number 725. Retrieved from http://www.thehorse.com/ViewArticle.aspx?ID=725
The acceptable disease?. (2010, 30 Dec.). Bringing the light to HYPP. Retrieved from http://www.bringinglighttohypp.org/BRIEF

### SUMMARY OF INVENTION

Candy Cakes allow the horse owner to simply give the horse a tasty treat, in lieu of a syringe full of light corn syrup. This treat is designed specifically with HYPP affected horses in mind, and each treat contains a measured amount of light corn syrup. In addition to being used for treatment of attacks, they can be used every day as part of a preventative plan or whenever the owner wishes to give the horse a treat. These treats are safe for all horses.

### BRIEF DESCRIPTION OF THE SEVERAL VIEWS OF THE DRAWING

Not applicable

### DETAILED DESCRIPTION OF THE INVENTION

The treats are a baked good, made of rolled oats and light corn syrup, with whole wheat flour used to help bind it together. The treats will contain a minimum of 30 cc's of light corn syrup per treat for large sized treats, and 20 cc's for smaller treats. Each treat is mixed individually to ensure the correct amount of light corn syrup for each one. The shape and size of the treat resembles a cup cake. Treats are baked in an oven at 425 degrees, with large treats baking around 35 minutes and small treats baking around 20 minutes. The treats may be available in a variety of flavors, simply by adding carefully selected ingredients and flavorings to the original formula. Toppings may be added as well. Additives include things such as carrots, apples, peppermint, or marshmallow, but they are not limited to that. The main design and usage will remain the same regardless of flavor additives. Usage suggestions are 2 to 3 treats, depending on size, to help stop a mild attack. This may change as the owner sees fit, based on the severity of the attack and the response from the horse. The owner may also treat the horse 1-2 treats at a time as part of a preventative plan, as well as use it as a treat whenever they wish. This treat contains no medications and should not be used as the sole prevention of attacks. It should be used in conjunction with veterinary instructions. The treat simply replaces the previous method of giving an HYPP horse light corn syrup via syringe.

### DRAWINGS

Not applicable

### SEQUENCE LISTING

Not applicable

1. The most important part of this application is the new process of using the treat to deliver a specific amount of light corn syrup, specifically for treating horse afflicted with HYPP.
